(19) United States
(12) Patent Application Publication
(10) Pub. No.: US 2003/0050302 A1
(43) Pub. Date: Mar. 13, 2003

(54) Treatment of conditions associated with amyloid processing using PKC activators

(75) Inventors: Rene Etcheberrigaray, Columbia, MD (US); Lixin Qiao, Arlington, VA (US); Alan P. Kozikowski, Princeton, NJ (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(73) Assignee: Neurologic, Inc.

(21) Appl. No.: 10/254,916

(22) Filed: Sep. 26, 2002

Related U.S. Application Data
(62) Division of application No. 09/652,656, filed on Aug. 31, 2000, now abandoned.

(51) Int. Cl. A61K 31/55

(52) U.S. Cl. 514/212.07

(57) ABSTRACT

A method for increasing the generation of non-amyloidogenic soluble APP comprising activation of protein kinase C (PKC) by administering an effective amount of at least one PKC activator. Also provided is a method for altering conditions associated with amyloid processing in order to enhance an α-secretase pathway to generate soluble α-amyloid precursor protein (α-APP) so as to prevent β-amyloid aggregation comprising administering an effective amount of a benzolactam.

### PRIORITY OF INVENTION

This application claims priority under 35 U.S.C. §119(e) from U.S. Provisional Applications No. 60/151,626, filed Aug. 31, 1999, Number 60/194,861, filed Apr. 6, 2000, and is a divisional application of application Ser. No. 09/652,656, hereby incorporated by reference.

### BACKGROUND OF THE INVENTION

1. Field of the Invention

The present invention relates to compounds for treatment of conditions associated with amyloid processing such as Alzheimer's Disease and compositions for the treatment of such conditions. The invention further relates to compounds for the treatment of conditions associated with amyloid processing.

2. Description of the Related Art

Kozikowski et al. in International Application No. PCT/US97/08141, discloses certain compounds for the modulation of protein kinase C (PKC). Protein kinases serve a regulatory function which is crucial for all aspects of cellular development, differentiation and transformation. PKC was identified as one of the largest gene families of non-receptor serine-threonine protein kinases. Since the discovery of PKC in the early eighties by Nishizuka and coworkers (Kikkawa et al., J. Biol. Chem., 257, 13341 (1982), and its identification as a major receptor for phorbol esters (Ashendel et al., Cancer Res., 43, 4333 (1983)), a multitude of physiological signaling mechanisms have been ascribed to this enzyme. The intense interest in PKC stems from its unique ability to be activated in vitro by diacylglycerol (and its phorbol ester mimetics), an effector whose formation is coupled to phospholipid turnover by the action of growth and differentiation factors.

Kozikowski et al., in PCT/US97/08141, provided certain benzolactam PKC modulators exhibiting PKC isoform selectivity. The application teaches that pharmaceutical compositions comprising an amount of such benzolactam modulators were effective to treat mammalian conditions associated with pathological cellular proliferation, particularly human cancers such as solid tumors and leukemias. The application further teaches a method to inhibit the pathological proliferation of mammalian cells, such as cancer cells, by administering to a mammal afflicted with such a condition, an effective inhibitory amount of one or more of the benzolactam modulators.

Shudo et al., U.S. Pat. No. 5,652,232, discloses benzolactam derivatives represented by the formula:

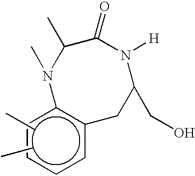

According to Shudo et al., the benzolactam derivatives had excellent inhibitory activities against retroviruses and reduced side effects such as cytotoxicity. The derivatives were purported to be useful for the treatment and prevention of AIDS.

Compounds in addition to benzolactams have been found to have a modulating effect on PKC. For example, PKC can be activated by phorbol esters which significantly increase the relative amount of non-amyloidogenic soluble APP (sAPP) secreted. Activation of PKC by phorbol ester does not appear to result in a direct phosphorylation of the APP molecule, however. Irrespective of the precise site of action, phorbol-induced PKC activation results in an enhanced or favored α-secretase, non-amyloidogenic pathway. Potentially then, PKC activation could be an attractive approach to influence the production of non-deleterious and even beneficial sAPP and at the same time reduce the relative amount of Aβ peptides.

Phorbol esters, however, may not be suitable compounds for eventual drug development because of their tumorigenic activity.

### SUMMARY AND OBJECTS OF THE INVENTION

The present invention relates to a novel PKC activator that shows antitumoral activity and possesses improved isozyme selectivity, and effectively enhances the secretion of sAPP inhuman fibroblasts from AD patients and in PC12 cells. A related smaller membered, synthetic compound (LQ12) can also enhance sAPP secretion in PC-12 cells.

In a first aspect, the present invention relates to a method for treating Alzheimer's disease comprising administering to a subject an effective amount of a benzolactam.

In a second aspect, the present invention relates to a method for altering conditions associated with amyloid processing in order to enhance an α-secretase pathway to generate soluble α-amyloid precursor protein (α-APP) so as to prevent β-amyloid aggregation comprising administering a biologically effective amount of a benzolactam.

In a third aspect, the present invention relates to a composition for treating Alzheimer's disease comprising:

(I) a benzolactam in an amount effective to generate soluble α-APP and prevent β-amyloid aggregation;

(II) a pharmaceutically effective carrier.

The benzolactam is preferably (2S, 5S)-8-(1′-decynyl)benzolactam or (2S, 5S)-8-(1′-decynyl)-7-methoxylbenzolactam.

### BRIEF DESCRIPTION OF THE DRAWINGS

FIG. 1 sets forth (A) fluorescent images of human fibroblasts in culture at subconfluent levels; (B) TEA response profile in AD cells;

FIG. 2 sets forth a bar graph depicting Calcium elevations in response to TEA expressed as a percentage of responding cells;

FIG. 3 depicts cell-attached recordings in human fibroblasts;

FIG. 4 depicts (A) representative immunoblots from an AC and an AD cell line treated with BL for one minute and (B) a bar graph depicting PKC redistribution from cytosol (soluble fraction) to membrane (particulate fraction) expressed as a ratio of particulate to soluble fraction immunoreactivity measured by densitometric analyses in seven AD cell lines;

FIG. 5 is a bar graph depicting benzolactam induced secretion of sAPP in human fibroblasts;

FIG. 6 depicts immunoblots of sAPP secreted by AC and AD. Representative Western blots show the enhanced secretion of sAPP by BL in AD and AC fibroblasts;

FIG. 7 is a bar graph depicting PDBu-induced secretion of sAPP in human fibroblasts. PDBu induced significant elevations of sAPP in AD (solid bars); and

FIG. 8 is a bar graph depicting the effects of BL and LQ-12 on sAPP secretion in PC-12, wherein densitometric values of sAPP on treated cells were compared with untreated controls for each experiment and repeated four times.

### DETAILED DESCRIPTION OF PREFERRED EMBODIMENTS

The benzolactams which may be employed according to the present invention include those disclosed in PCT/US97/08141, the disclosure of which is hereby incorporated by reference. These benzolactams are of general formula:

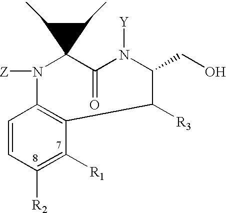

wherein R₁ is H, (C₁-C₅)alkyl, OR^{a}, SR^{a}, N(R^{a})(R^{b}), halo, NO₂, NHC(O)[(C₁-C₄)alkyl] or NHOH;

R₂ is a (C₅-C₂₂) hydrocarbyl group, optionally comprising 1-3 double bonds, 1-2 triple bonds or a mixture thereof, or (C₆-C₁₂)aryl(C₂-C₁₀)alkyl, wherein the alkyl moiety optionally comprises 1-2 double bonds, 1-2 triple bonds or a mixture thereof; wherein said (C₅-C₂₂) hydrocarbyl group or said (C₆-C₁₂)aryl(C₁-C₁₀)alkyl may optionally be substituted with 1 or 2 substituents independently selected from the group consisting of halo, hydroxy, cyano, nitro, (C₁-C₅)alky, (C₁-C₅)alkoxy, trifluoromethyl, trifluoromethoxy, —C(═O)O(C₁-C₅)alkyl, and N(R^{c})(R^{f});

R₁ and R₂ together are —CH(R^{c})—CH₂—C(O)—N(R_{d})—, —C(R^{c})═CH—CC(O)N(R^{d})—,—C(R^{c})═CH—O—;

R₃ is H, OH or halo;

R^{a} and R^{b} are independently H or (C₁-C₅)alkyl;

R^{c} is a (C₅-C₂₂)hydrocarbyl group;

R^{d} is H or (C₁-C₅)alkyl;

R^{e} and R^{f} are independently hydrogen, (C₁-C₅)alkyl, or (C₁-C₅)alkanoyl, or together with the nitrogen to which they are attached are pyrrolidino, piperidino or morpholino;

Z is H or (C₁-C₅)alkyl; and

Y is H or (C₁-C₅)alkyl; or a pharmaceutically acceptable salt thereof.

Two preferred benzolactams include (2S, 5S)-8-(1′-decynyl)benzolactam or (2S, 5S)-8-(1′-decynyl)-7-methoyxlbenzolactam.

The present inventors have studied benzolactams as activators of protein kinase (PKC). Alterations in PKC, as well alterations in calcium regulation and potassium (K⁺) channels are included among alterations in fibroblasts in Alzheimer's disease (AD) patients. Since PKC is known to regulate ion channels, the present inventors have studied K⁺ channel activity in fibroblasts from AD patients in the presence of (2S, 5S)-8-(1′-decynyl)benzolactam (BL), a novel activator of PKC with improved selectivity for the α, β, and γ isoforms. Restoration of normal K⁺ channel function, as measured by TEA-induced [Ca²⁺] elevations, occurs due to activation of PKC by BL. Representative patch-clamp data further substantiate the effect of BL on restoration of 113 pS K⁺ channel activity. Immunoblotting analyses using an α-isozyme-specific PKC antibody confirm that BL-treated fibroblasts of AD patients show increased PKC activation. Thus PKC activator-based restoration of K⁺ channels offers another approach to the investigation of AD pathophysiology, thereby providing a useful model for AD therapeutics.

The use of peripheral tissues from Alzheimer's disease (AD) patients and animal neuronal cells permitted the identification of a number of cellular/molecular alterations that may be the reflection of comparable processes in the AD brain and thus, of pathophysiological relevance (Baker et al., 1988; Scott, 1993; Huang, 1994; Scheuner et al., 1996; Etcheberrigaray & Alkon, 1997; Gasparini et al., 1997). Alterantiosns of potassium channel function have been identified in fibroblasts (Etcheberrigaray et al., 1993) and in blood cells (Bondy et al., 1996) obtained from AD patients. In addition, it was shown that β-amyloid, widely accepted as a major player in AD pathophysiology (Gandy & Greengard, 1994; Selkoe, 1994; Yankner, 1996), was capable of inducing an AD-like K⁺ channel alteration in control fibroblasts (Etcheberrigaray et al., 1994). Similar or comparable effects of β-amyloid on K⁺ channels have been reported din neurons from laboratory animals (Good et al., 1996; also for a review see Fraser et al., 1997). An earlier observation of hippocampal alterations of apamin-sensitive K⁺ channels in AD brains (as measured by apamin binding) provides additional support for the suggestion that K⁺ channels may be pathophysiologically relevant in AD (Ikeda et al., 1991). Furthermore, protein kinase C (PKC) exhibits parallel changes in peripheral and brain tissues of AD patients. The levels and/or activity of this enzyme(s) were introduced in brains and fibroblasts from AD patients (Cole et al., 1988; Van Huynh et al., 1989; Govoni et al., 1993; Wang et al., 1994). Studies using immunoblotting analyses have revealed that of the various PKC isozymes, primarily the a isoform was significantly reduced in fibroblasts (Govoni et al., 1996), while both a and P isoforms are reduced in brains of AD patients (Shimohama et al., 1993; Masliah et al., 1990). These brain PKC alterations might be an early event in the disease process (Masliah et al., 1991). It is also interesting to note that PKC activation appears to favor nonamyloidogenic processing of the amyloid precursor protein (APP; Buxbaum et al., 1990; Gillespie et al., 1992; Selkoe, 1994; Gandy & Greengard, 1994; Bergamashi et al., 1995; Desdouits et al., 1996; Efhimiopoulus et al., 1996). Thus, both PKC and K⁺ channel alterations appear to coexist in AD, with peripheral and brain expression in AD.

The link between PKC and K⁺ channel alterations was investigated. Because PKC is known to regulate ion channels, including K⁺ channels (e.g., see Alkon et al., 1988; Covarrubias et al., 1994; Hu et al., 1996), a defective PKC leads to defective K⁺ channels. To demonstrate this, AD fibroblasts were used in which both K⁺ channels and PKC defects had been independently demonstrated (Etcheberrigaray et al., 1993; Govoni et al., 1993, 1996). Fibroblasts with known dysfunctional K⁺ channels were treated with PKC activators and restoration of channel activity was monitored as presence/absence of TEA-induced calcium elevations. An assay based on tetraethylammonium chloride (TEA)-induced [Ca²⁺] elevation was used because it has been shown to depend on functional 113 pS K⁺ channels that are susceptible to TEA blockade (Etcheberrigaray et al., 1993, 1994; Hirashima et al., 1996). Thus, TEA-induced [Ca²⁺] elevations and K⁺ channel activity are primarily observed in fibroblasts from control individuals while being virtually absent in fibroblasts from AD patients (Etcheberrigaray et al., 1993; Hirashima et al., 1996).

It was thus demonstrated that the use of a potent novel PKC activator, benzolactam (BL), restored the responsiveness of AD fibroblast cell lines to the TEA challenge. Immunoblot evidence demonstrates that this restoration is related to a preferential participation of the a isoform since BL shows improved selectivity for this isozyme that is defective in AD fibroblasts.

The present inventors have also observed that activation of protein kinase C favors the α-secretase processing of the Alzheimer's disease (AD) amyloid precursor protein (APP), resulting in the generation of non-amyloidogenic soluble APP (sAPP). Consequently, the relative secretion of amyloidogenic Aβ₁₋₄₀ and Aβ₁₋₄₂₍₃₎ is reduced. This is particularly relevant since fibroblasts and other cells expressing APP and presenilin AD mutations secrete increased amounts of total Aβ and/or increased ratios of Aβ₁₋₄₂₍₃₎/Aβ₁₋₄₀. Interestingly, PKC defects have been found in AD brain (α and β isoforms) and in fibroblasts (α-isoform) from AD patients. Here, we use a novel PKC activator (benzolactam, BL) with improved selectivity for the α, β and γ isoforms to enhance sAPP secretion over basal levels. SAPP secretion in BL-treated AD cells was also slightly higher compared to control BL-treated fibroblasts, which only showed significant increases of sAPP secretion after treatment with 10 μM BL. Staurosporine (a PKC inhibitor) eliminated the effects of BL in both control and AD fibroblasts. BL and a related compound (LQ13) also cause a ˜3-fold sAPP secretion in PC12 cells. The use of a novel and possibly non-tumorigenic PKC activator may prove useful to favor non-amyloidogenic APP processing and is, therefore, of potential therapeutic value.

The processing of the amyloid precursor protein (APP) determines the production of fragments that later aggregate forming the amyloid deposits characteristic of Alzheimer's disease (AD), known as senile or AD plaques. Thus, APP processing is an early and key pathophysiological event in AD.

Three alternative APP processing pathways have been identified. The previously termed “normal” processing involves the participation of an enzyme that cleaves APP within the Aβ sequence at residue Lys16 (or between Lys16 and Leu17; APP₇₇₀ nomenclature), resulting in non-amyloidogenic fragments: a large N-terminus ectodomain and a small 9 kDa membrane bound fragment. This enzyme, yet to be fully identified, is known as α-secretase. Two additional secretases participate in APP processing. One alternative pathway involves the cleavage of APP outside the Aβ domain, between Met671 and Asp672 (by β-secretase) and the participation of the endosomal-lysomal system. An additional cleavage site occurs at the carboxyl-terminal end of the Aβ portion, within the plasma membrane after amino acid 39 of the Aβ peptide. The secretase (γ) action produces an extracellular amino acid terminal that contains the entire Aβ sequence and a cell-associated fragment of 6 kDa. Thus, processing by β and γ secretases generate potential amyloidogenic fragments since they contain the complete Aβ sequence. Several lines of evidence have shown that all alternative pathways occur in a given system and that soluble Aβ may be a “normal product.” However, there is also evidence that the amount of circulating Aβ in CSF and plasma is elevated in patients carrying the “Swedish” mutation. Moreover, cultured cells transfected with this mutation or the APP₇₁₇ mutation secrete larger amounts of Aβ. More recently, carriers of other APP mutations and PS1 and PS2 mutations have been shown to secrete elevated amounts of a particular form, long (42-43 amino acids) Aβ.

Therefore, although all alternative pathways may take place normally, an imbalance favoring amyloidogenic processing occurs in familial and perhaps sporadic AD. These enhanced amyloidogenic pathways ultimately lead to fibril and plaque formation in the brains of AD patients. Thus, intervention to favor the non-amylodoigenic, α-secretase pathway effectively shifts the balance of APP processing towards a presumably non-pathogenic process that increases the relative amount of sAPP compared with the potentially toxic Aβ peptides.

The following examples are given by way of illustration and should in no way be construed as limiting the general disclosure of the invention.

### EXAMPLE 1

Materials and Methods

Cell Culture

Cultured skin fibroblasts were obtained from the Coriell Cell Repositories and grown using the general guidelines established for their culture with slight modifications (Cristofalo & Carptentier, 1988; Hirashima et al., 1996). The culture medium in which cells were grown was Dulbecco's modified Eagle's medium (GIBCO) supplemented with 10% fetal calf serum (Biofluids, Inc.). Seven age-matched controls (AC) (AG06241, AG07141, AG05266, AG09878, AG04560, AGO6010, and AG08044), three sporadic AD (SAD cases (AG06263, AG07375, and AG07377), and four familian AD (FAD) cases-including Canadian family 964 (Nee et al., 1983) (AG06848, AG08170, Italian family 1079 (AG07872), and pedigree 747 (AG04401)-were tested in the calcium-imaging experiments. Mutations in the PSI gene are expressed in both the Canadian (an Ala→Glu substitution in codon 246; PS 1A246E) and the Italian (an Met→Leu substitution at codon 146; PS1M146L) family cell lines. For immunoblotting experiments, the same set of seven AC and AD cell lines tested in the imaging experiments were used. Detailed information about these cell lines can be obtained elsewhere (Nee et al., 1983; National Institute of Aging, 1994).

PKC Activators

The different tissue distributions, the apparently distinctive roles of different isozymes, and the differential involvement in pathology make it important to use pharmacological tools that are capable of preferentially targeting specific isozymes (Kozikowski et al., 1997; Hofmann, 1997). Recent research in the medicinal chemistry field has resulted in the development of non-tumor-promoter PKC activators that target with greater specificity the a and β isoforms (Kozikowski et al., 1997). One such compound is (2S, 5S)-8-(1′-decynyl)-benzolactam (benzolactam), a derivative of the natural product indolactam V. Indolactam V was first isolated from the mycelia of Streptomyces mediocidicus and is a member of the teleocidin family. These compounds, like the phorbols, compete for the regulatory domain of PKC and engage in very specific hydrogen bond interactions within this site. Additional information on the organic chemistry and molecular modeling of this compound can be found elsewhere (Kozikowski et al., 1997). For comparison purposes, we also used the nonselective well-known PKC activator, phorbol 12,13-dibutyrate.

Calcium Imaging

Round, 25-mm glass coverslips were flame-polished and placed inside 35-mm Nunc petri dishes. Cells were seeded (ca. 10-15 cells/mm²) in these petri dishes and used when cell density was equivalent for all cell lines (ca 180 cells/mm²). General aspects of the calcium-imaging technique and the theoretical foundation are described elsewhere (Thomas et al., 1991). Briefly, culture medium was removed and cells were washed at least three times with basal salt solution (BSS; in mM: NaCl 140, KCl 5, CaCl₂ 2.5, MgCl₂ 1.5, Hepes 10, glucose 5, pH 7.4). The fluorescent probe was loaded by incubating cells in 1 μM fura 2-AM (Molecular Probes) in 1 ml BSS at room temperature (RT). One hour later, cells were washed thoroughly with BSS and 1 ml of fresh BSS was added for [Ca²⁺] baseline measurements. Fluorescent images at 334 and 380 nm were acquired so that the rate of ratios was approximately 1/s to about 300/s with a Zeiss-Attofluor Ratio Arc Imaging System (Zeiss). A 40× Zeiss Fluor (oil immersion) objective lens was used.

Treatment

Cells from all treatment groups were tested with 100 mM TEA (Sigma) by applying 3 ml of TEA-modified BSS (in mM: TEA 133.3, NaCl 6.7, KCl 5, CaCl₂ 2.5, MgCl₂ 1.5, Hepes 10, glucos 5, pH 7.4) to the dish. TEA was dissolved in BSS and the solution was accordingly modified to prevent osmolarity changes (Hirashima et al., 1996). A group of cells was treated with the PKC activator benzolactam prior to the TEA challenge. BL was dissolved in dimethyl sulfoxide (DMSO; Sigma) to achieve a final testing concentration of 50 nM when 1 ml of the dissolved BL was added to the dish 1 minute prior to TEA application. A second group of cells was incubated with the nonselective PKC activator, phorbol 12, 13 dibutyrate (PDBu; Calbiochem) at 50 nM for 45 minutes by adding the compound (dissolved in DMSO) to the dish 15 minutes after the initiation of the dye-loading phase. A preliminary study also included 1 and 15 minute incubation with PDBu. The control group of cells included cells which received no treatment prior to TEA challenge (untreated), cells pretreated with 50 nM DMSO for 1 minute, and cells incubated with 50 nM 4α-phorbol (4α-PHR; Calbiochem), an inactive form of phorbol ester (negative control), for 45 minutes. Seven to ten petri dishes of cells were tested from each cell line for each experimental condition. In almost all the cell lines, a minimum of 100 cells were tested per treatment per cell line (total 9231 cells). There were only three AC cell lines for which the number of cells in a couple of treatment groups was above 65 but below 100. Experiments for each cell line were repeated on at least one separate occasion (minimum 1-week interval). Response in a given cell was characterized as a double-fold increase in baseline [Ca²⁺] (75-90 nMO based on previously established criteria (Etcheberrigaray et al., 1993; Hirashima et al., 1996).

Electrophysiology and Single-Channel Analysis

Patch-claim experiments were performed on 13 separate replicate representative experiments in a well-characterized AD cell line (AG06848) which is normally silent for the 113 pS TEA-sensitive K⁺ channel (Etcheberrigaray et al., 1993). The experiment was performed at room temperature (21-23° C.) following standard procedures (Sakmann & Neher, 1983). The culture medium was replaced with the following solution (mM, NaCl 150, KCl 5, CaCl₂ 2, MgCl₂ 1, Hepes (NaOH) 10, pH 7.4, prior to recording. Benzolactam was added to a dish after ensuring that the patch is silent for the II 3 pS K⁺ channel. The recording electrodes, filled with a high-K⁺ solution (mM), KCl 140, CaCl₂ 2, MgCl₂ 1, Hepes (NaOH) 10, pH 7.4, were made from Blu Tip capillary tubes (i.d. 1.1-1.2 mm; Oxford Labware) by using a BB-CH-PC Mecanex puller to obtain pipette resistances that were ˜6 MΩ. An Axopath-IC amplifier was used to obtain records and a JVC Vetter PCM videorecorder was used to store the data on tape. The data were acquired and analyzed on a personal computer with a pClamp suite of programs by transferring the information via an Axolab interface from the amplifier. All three components-amplifier, interface, and software-were obtained from Axon Instruments (Foster City, Calif.).

Immunoblot Assay

Immunoblot experiments were conducted using well-established procedures (Dunbar, 1994). Cells were grown to confluency (˜90%) in t-75 flasks. Levels of a isozyme in response to treatment with 50 nM BL for 1 and 15 minutes or 50 nM PDBu for 1 and 45 minutes were quantified using procedures slightly modified from that established by Racchi et al., (1994). Fibroblasts were washed twice with ice-cold PBS, scraped in PBS, and collected by low-speed centrifugation. The pellets were resuspended in the following homogenization buffer: 20 mM Tris-HCl, pH 7.5, 2 mM EDTA, 2 mM EGTA, 5 mM DTT, 0.32 M sucrose, 2 mM PMSF, 25 μg/ml leupeptin. Homogenates were immersed in liquid nitrogen, thawed, and centrifuged at 12,000 g for 20 minutes, and the supernatants were used as the cytosolic fraction. The pellets were homogenized in the same buffer containing 1.0% Triton X-100, incubated in ice for 45 minutes, and centrifuged at 12,000 g for 30 minutes. The supernatant from this batch was used as the membranous fraction. After protein determination, 20 μg of protein was diluted in 2× electrophoresis sample buffer (Novex), boiled for 5 minutes, run on 12% SDS-PAGE, and transferred electrophoretically to nitrocellulose membrane. The membrane was saturated with Blotto blocker (Pierce) by incubating it at RT for an hour. The primary antibody for PKC α isoform (Transduction Laboratories) was diluted (1:1000) in blocking solution and incubated with the membrane for 1 hour at RT. After incubation with the secondary antibody, alkaline phosphatasae antimouse IgG (Vector Laboratories), the membrane was developed using an alkaline phosphatase kit (Vector Laboratories) as per the manufacturer's instructions. The conditions for developing the membranes were kept as similar as possible. After a digital image of each nitrocellulose membrane was produced (Lightools Research Speedlight Gel Documentation System), although the OD value (by performing densitometric analyses; NIH Image version 1.6) for each signal was obtained, the ratio of membrane to particulate PKC fraction was used when comparing among experiments to avoid idiosyncratic variability. The assay was repeated at least twice for all cell lines except two AC (AG05266, AG09878) and two AD (AG07872, AG07375) cell lines. The data from each replicate were pooled for each cell line and averaged across cell lines for each treatment group.

Statistical Analyses

Data were pooled from FAD and SAD cell lines since initial analyses revealed no significant differences in response patterns between the two types of cell lines in both imaging and immunoblotting studies. All imaging and immunoblot data analyses were performed using the two-tailed t test (Remington & Schork, 1995) and, in cases in which the variances were different, using the Welch correction (Motulsky, 1995) in Graph Pad Prism (version 2) for IBM. The sole exception was the one-tailed t test statistical comparison to evaluate whether the level of PKC immunoreactivity after 1 minute treatment with BL is stronger than the 1 minute treatment with PDBu.

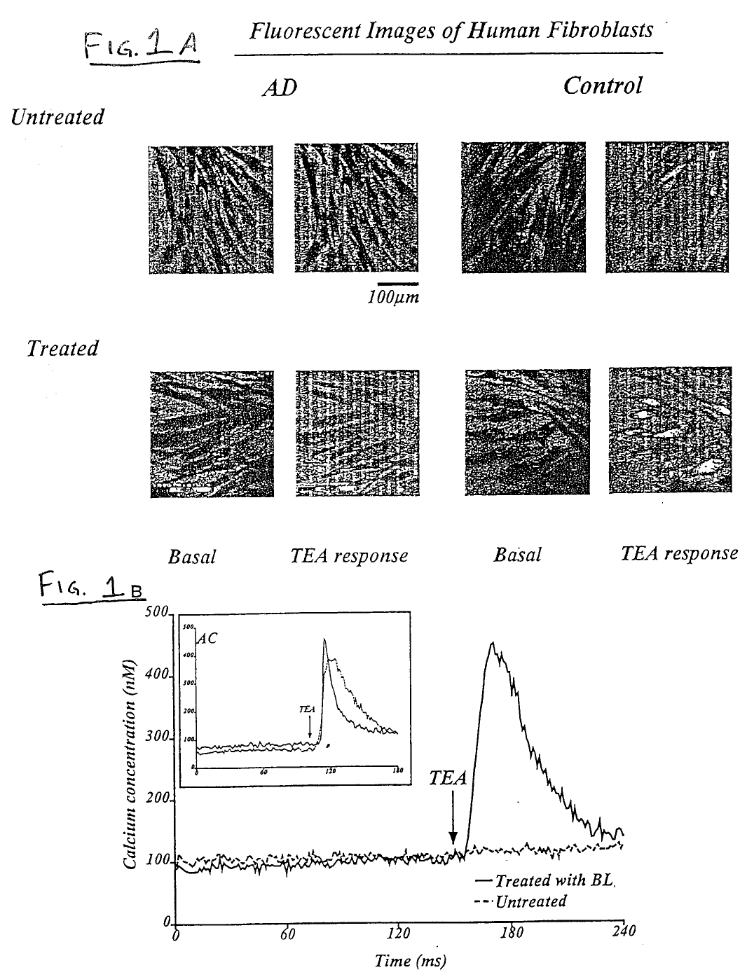

FIG. 1(A) depicts fluorescent images of human fibroblasts in culture at subconfluent levels. Pseudocolor ratio images represent calcium levels as seen when cells are excited by 334 nm (numerator) and 380 nm (denominator) UV light. Brighter ratio images depict intracellular increase in calcium levels. While there is no difference in calcium levels in untreated AD cells before and after TEA application, AD cells pretreated fro 1 min with benzolactan (BL) respond to TEA with an increase in intracellular Ca²⁺ from basal levels. On the other hand, there is an increase in Ca²⁺ in both untreated and BL-pretreated AC cells following TEA challenge. FIG. 1B depicts the TEA response profile in AD cells. The lack of intracellular Ca²⁺ elevation following TEA-application in untreated AD cells is reversed in BL-pretreated AD cells. The inset in FIG. 1B shows how TEA application results in an increase in Ca levels in both untreated and BL-pretreated AC cells. Lines represent the same treatment as in FIG. 1B.

Pseudocolor fluorescent ratio images of basal and TEA-induced calcium levels in fibroblasts excited by 334 (numerator) and 380 nm (denominator) UV light are shown in FIG. 1A. Pretreatment of AD fibroblasts were benzolactam (FIG. 1B, solid lines) restores the typical increase in intracellular calcium following TEA treatment in contrast to the untreated AD cells (broken line). On the other hand, [Ca²⁺] elevations occurred in both untreated and BL-pretreated AC cells, due to TEA-induced depolarization by blockade of 113 pS K⁺ channels (FIG. 1, inset)

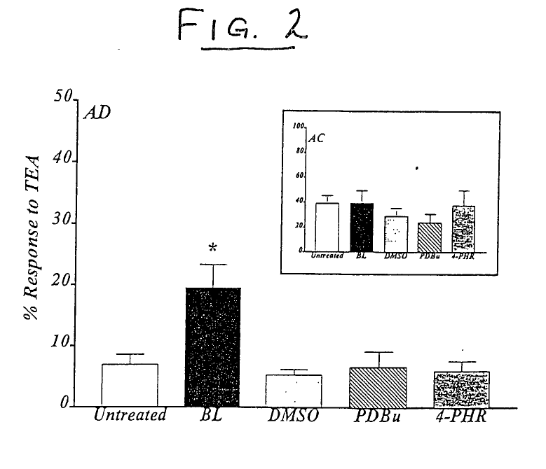

Following TEA challenge, the percentage of cells responding to TEA was measured and averaged across cell lines for AC and AD cases (FIG. 2). More particularly, FIG. 2 shows calcium elevation in response to TEA expressed as percentage of responding cells. Each of the cell lines received one of the following treatments-none (open bar; N=1067), one minute pretreatment with either BL (black bar; N=1038) or DMSO (light gray bar; N=974), or 45 minute incubation with either PDBu (hatched bar; N=1047) or 4α-phorbol (dark gray bar; N=1034). Data from each replicate within each cell line were pooled and averaged (means±s.d.) across the seven AD cell lines for each treatment. Benzolactam pretreatment significantly increased the TEA responsiveness compared to untreated or DMSO-treated controls (*P<0.05 in either case). In contrast, there was no difference between PDBu and 4α-PHR-pretreated AD cells (ns). Two-tailed t test was used to make statistical comparisons. Inset: Normal response to TEA in AC cells irrespective of treatment-none (open bar; N=887), pretreatment with BL (black bar; N=824), DMSO (light gray bar; N=754), PDBu (hatched bar; N=794), or 4α-phorbol (dark gray bar; N=812). Neither BL-treated AC cells differed from untreated or DMSO-treated cells in percentage of cells responding to TEA nor PDBu-treated cells from their respective control, the 4-PHR-treated AC cells. Percentage of cells responding to TEA is significantly higher in untreated AC versus untreated AD cells (P<0.005).

Upregulating PKC (predominantly the a isoform) in AD fibroblasts restored TEA response. Treatment of AD cell lines with 50 nM BL for 1 minute (black bar) significantly increased the percentage of cells responding to TEA compared to the untreated (open bar; t=2.99, P=0.017) and DMSO-treated (light gray bar; t=3.60, P=0.0113) controls. In contrast, AD cells incubated with 50 nM nonselective PKC activator, PDBu, for 45 minutes did not differ in their TEA response (hatched bar) from the 4α-PHR-treated cells (dark gray bar; t=0.22, P=0.83). Shorter incubation times (1 and 15 minutes) with PDBu were also without effect (data not shown). Typical TEA response was observed in all AC cells irrespective of treatment (FIG. 2). Percentage of response in BL-treated cells did not differ from untreated (t=0.02, P=0.98) or DMSO-treated (t=0.88, P=0.39) AC cells. Likewise, percentage of PDBu-treated cells responding to TEA was not different from the 4α-PHR-treated (t=1.01, P=0.33) AC cells. The significantly higher percentage of “responsive” untreated AC (39.19+1.75) cells (open bars in main graph and insert; t=4.97, P=0.0025) is consistent with previous findings (Etcheberrigaray et al., 1993; Hirashima et al., 1996).

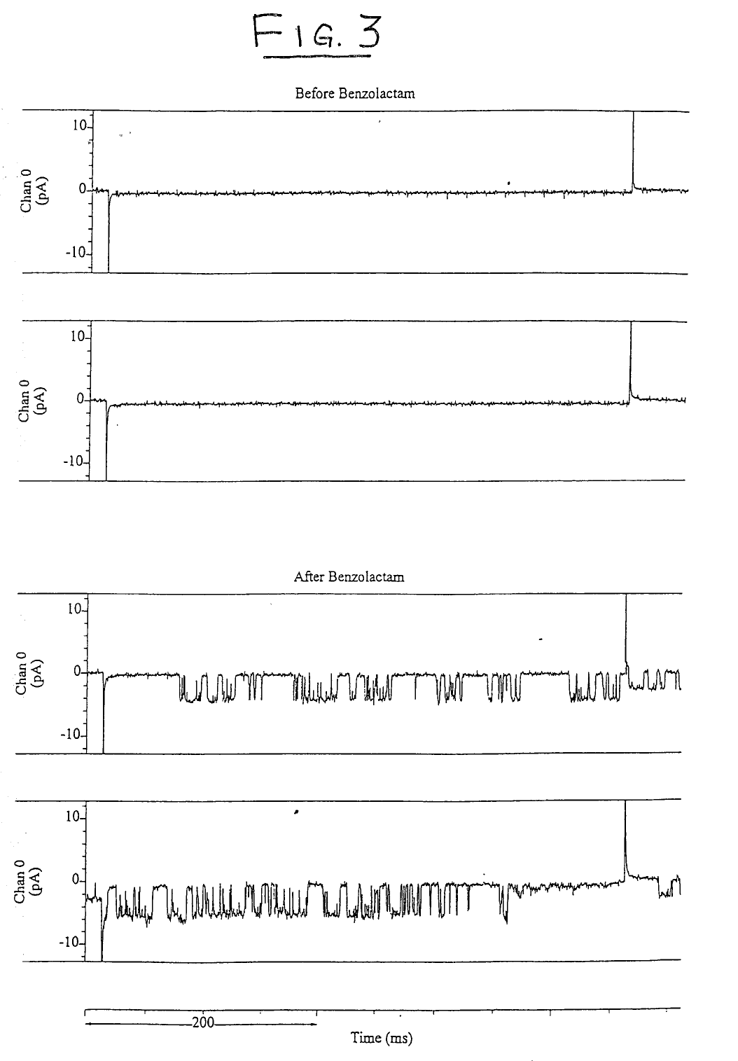

Referring now to FIG. 3, there is shown representative traces obtained from the cell line AG06848 (FAD). The top two traces correspond to a silent patch at 0 and +20-mV pipette potentials. Distinct channel activity (bottom two traces) was observed in the same patch after adding a PKC activator, benzolactam, to the dish.

Patch-clamp analyses revealed that none of the 13 recordings showed K⁺ channel activity before application of BL to the medium (see FIG. 3, top two panels). However, after 1 minute BL treatment, 7 of the same 13 cells showed unambiguous 113 pS K⁺ channel (see FIG. 3, bottom two panels). The 54% K⁺ channel activity after BL activation in these AD cells is comparable to that observed in control cells in a previous study (Etcheberrigaray et al., 1993).

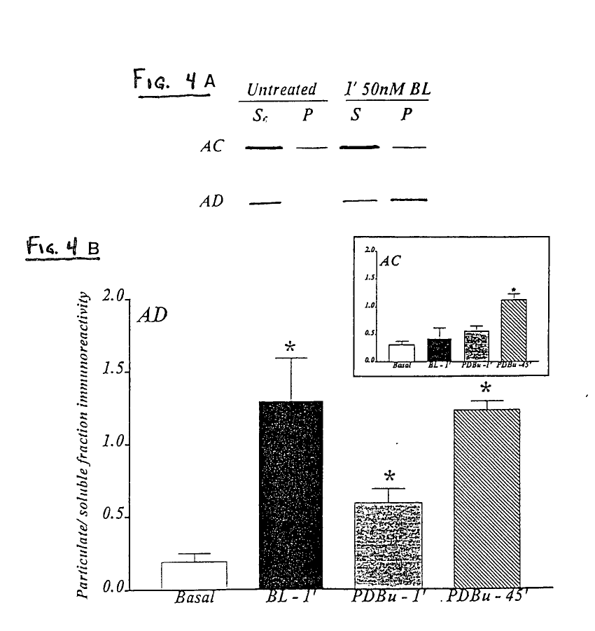

Immunoblot assay provides an additional support for our hypothesis that PKC is involved in the restoration of TEA response. FIG. 4 demonstrates the translocation of PKCα induced by benzolactam. FIG. 4(A) sets forth representative immunoblots from an AC and an AD cell line treated with BL for 1 minute. FIG. 4(B) sets forth PKC redistribution from cytosol (soluble fraction) to membrane (particulate fraction) expressed as ratio of particulate to soluble fraction immunoreactivity measured by densitometric analyses in seven AD cell lines. Ratio values for untreated, 1 minute treatment with BL or PDBu, and 45 minute pretreatment with PDBu are represented by open, black, gray, and hatched bars, respectively. Data from each replicate were pooled within each cell line and averaged (means±s.d.) across cell lines. Benzolactam treatment of AD cells significantly increased the level of immunoreactivity from basal levels (*P<0.05). Incubation with PDBu for 1 or 45 minutes also enhanced the level of enzyme activity from basal level (*P<0.05). Two=tailed t test was used for statistical comparisons. Inset: PKC immunoreactivity in AC cells. Data were averaged across seven AC cell lines after pooling replicates within each cell line. Bars represent the same treatments as in the main graph. Benzolactam or PDBu treatment for 1 minute did not have an effect on immunoreactivity of the enzyme in AC cells. However, incubation with PDBu for 45 minutes significantly increased the level of enzyme activity compared to the basal level (*P<0.05; two-tailed t test).

Compared to the basal ratios (FIG. 4B, open bar), treatment of AD cell lines with 50 nM BL for 1 minutes (FIG. 4B, black bar) revealed a pronounced redistribution of the a isoform of the enzyme from cytosol to membrane (t=3.67; P<0.05). A longer incubation time (15 minutes; data not shown) with BL further increased the particulate/soluble ratios (ratio=1.156; t=4.48; P<0.005) compared to basal levels, indicating lack of downregulation within the time frame studied. Although 1 minute treatment with 50 nM PDBu (FIG. 4B, gray bar) yielded a significant change from the basal ratio level (t=3.67; P<0.005) in AD cells, it was not as effective as the 1 minute treatment with 50 nM BL (black vs. gray bar, t=2.27, P<0.05). Longer incubation (45 minutes) with PDBu (FIG. 4B, hatched bar) resulted in levels of PKC translocation comparable to that observed with 1 minute BL treatment in AD cells (t=10.52; P <0.001).

In AC cells, effect of BL on enzyme translocation was significant only after 15 minute treatment with the PKC activator (data now shown; ratio =1.72; t=10.37; P<0.01) and not after 1 minute treatment (FIG. 4, black tar, t=0.56, ns) compared to basal levels (open bar). Likewise, long-term incubation (45 minutes) with PDBu had an effect on redistribution of the enzyme from cytosol to membrane (hatched bar, t=6.78, P<0.001). There was no effect of 1 minute incubation with PDBu (gray bar, t=2.16, ns) on PKC translocation in AC cells.

These results demonstrate the role of PKC in AD and suggest a mechanistic explanation for the various molecular and cellular defects observed in fibroblasts from AD patients. Restoration of the TEA-induced [Ca²⁺] responses in BL-treated AD cells suggests restoration of their K⁺ channel function. Patch-clamp data strongly support the conclusions from imaging studies in a representative sample obtained from a well-characterized AD cell line. BL was chosen since it has been shown to have improved isozyme specificity for the α and β isozymes and, to a lesser extent, for the γ isoform (Kozikowski et al., 1997). This improved selectivity is of particular relevance in AD fibroblasts, in which only the a isoform appears to be affected. In fact, the use of a compound that primarily targets this isoform proved to be a successful strategy to restore K⁺ to channel function in fibroblasts. Moreover, this effect was not seen in cells treated with PDBu, a nonspecific and-at equimolar concentrations-less potent activator of PKC. Although BL also exhibits high selectivity for the P isoform, the participation of this isozyme in restoring K⁺ channel function can be ruled out since it is not expressed in fibroblasts (Racchi et al., 1994). We confirmed this observation in a subsample of the cell lines used here (not shown). The γ isoform is also not expressed in fibroblasts (Racchi et al., 1994). The isoforms δ and ε are detected in fibroblasts (Racchi et al., 1994) but the BL affinity for them is at least 10-fold lower than for α (Kozikowshi et al., 1997). Furthermore, they do not appear to be altered in AD fibroblasts (Govoni et al., 1996).

The percentage of untreated AC cells responding to TEA (39.19%) is comparable to response levels reported in a previous study (Etcheberrigaray et al., 1993). Interestingly, treating AD cells with BL enhanced the level of TEA responsiveness (19.58%) to levels reported for AC cells in previous studies (Hirashima et al., 1996). Lack of such enhancement in BL-treated Ac cells is, perhaps, due to the system reaching a steady-state equilibrium of activity in these cells.

The immunoblot assay, under conditions approximating the imaging studies as closely as possible, provided an independent method of assessing that PKC is, indeed, involved in the restoration of the normal K⁺ channel phenotype. At equimolar concentrations, the effect of BL treatment was clearly stronger at 1 minute compared to the effect of PDBu at 1 minute for both imaging and immunoblot studies. However, PDBu was able to induce a noticeable change in PKC translocation but not in TEA-induced Ca²⁺ elevation at a longer (45 minute) time interval. Perhaps this singular disparity between imaging and immunoblot results is due to the different dynamics between activation of PKC and TEA-induced Ca²⁺ elevations. Although our experimental design did not demonstrate a direct effect of PKC on the channels, the PKC changes are observed within a time frame and under experimental conditions compatible with a casual link. Whether PKC acts directly on the channel or via an intermediary remains to be elucidated.

It is also interesting to consider the data in relation to APP metabolism and the effects of its subproducts. Studies have demonstrated that PKC activation increases the amount of ratio of nonamyloidogenic (soluble APP, presumably product of the a secretase) vs. amyloidogenic (Aβ1-40 and/or Aβ-42) secreted fragments (Buxbaum et al., 1990; Gillespie et al., 1992; Selkoe, 1994). We could speculate that AD cells with low PKC would have an impaired secretion of sAPP and/or have increased proportion of amyloidogenic fragments. Indeed, there is evidence that some AD cell lines exhibit both defective PKC and impaired sAPP secretion (Bergamaschi et al., 1995; Govoni et al., 1996). A subset of those cells was shown to have defective TEA responses (i.e., defective K⁺ channels; Hirashima et al., 1996). Moreover, at least three of the FAD cell lines used in this study are from individuals demonstrated to carry presenilin 1 mutations (St. George-Hyslop et al., 1992; Sherrington et al., 1995; Tanzi et al., 1996) and also to have increased levels of β-amyloid₁₋₄₂ (Hardy, 1997; Scheuner et al., 1996). In addition, β-amyloid has been shown to induce an AD-like K⁺ channel defect in fibroblasts (Etcheberrigaray et al., 1994) and to block K⁺ currents in cultured neurons (Good et al., 1996). Therefore, we suggest a mechanistic link such that an isozyme-specific PKC defect may lead to abnormal APP processing that, among other possible deleterious effects, alters K⁺ channel function. Recent preliminary data also suggest that, perhaps in a vicious cyclical manner, β-amyloid in turn causes reductions of α-PKC (Favit et al., 1997).

In summary, the data suggest that the strategy to upregulate PKC function targeting specific isozymes restores normal K⁺ channel function. The restoration of K⁺ function may have direct consequences in terms of reducing the progression of the pathological process. These studies and such a fibroblast model could be expanded and used as tools to monitor the effect of compounds (BL, for example) that alter potential underlying pathological processes. Such compounds could then be used as bases for rational design of pharmacological agents for this disorder.

### EXAMPLE 2

Materials and Methods

Cells and Cell Culture Procedures

Human fibroblasts were obtained from the Coriell Cell Repositories (Camden N.J.). Four AD cell lines (AG06848, AG04401, AG07377, AG06263) and four age-matched control cell lines (AG07141, AG06241, AG08044, AG04560) were grown to confluence in T75 cell culture flasks. Additional information about these cell lines can be found elsewhere. The culture medium was DMEM supplemented with 10% serum (FBS, Gibeo). PC12 cells were obtained from the American Cell Culture Collection and grown to confluence in T75 flasks containing DMEM supplemented with 5% FPS (Gibeo), 10% horse serum (Biofluids) and 1% of a mixture of penicillin (5000 units/ml in G sodium) and streptomycin (5000 μg/ml).

sAPP Secretion

Complete culture medium was removed just prior to experiments. Upon replacement with serum-free medium, cells were treated for 2 h with BL (0.5, 1 and 10 μM, obtained from the Shanghai Institute of Organic Chemistry), BL plus 0.5 μM staurosporine (ST, Sigma) added 30 min prior to BL, and vehicle alone (dimethyl sulfoxide (DMSO) Aldrich). For comparative purposes, another group of cells was treated with either PDBu (0.5, 1 and 10 μM, from Calbiochem) or PDBu plus staurosporine. PC12 cells had an additional treatment with LQ12, a synthetic PKC activator, related to BL (obtained from the G. U. Drug Discovery Program laboratories). The chemical structure of these novel PKC activators is set forth below. BL is a derivative of the natural product indolactam. LQ12 is a smaller lactam ring (5-membered) analog of BL (8-membered).

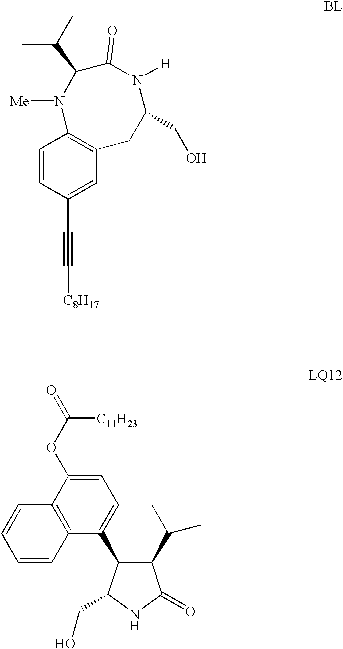

After two hours of incubation, the supernatant was removed and the secreted proteins were precipitated with 10% TCA and resuspended in Tris-glycine SDS electrophoresis sample buffer (Novex). The samples were boiled for 5 min, subjected to electrophoresis and later to immunoblotting for identification of sAPP.

Immunoblotting

Conventional immunoblotting techniques were employed. Protein extracts from each cell line and condition were loaded in precast 12% acrylamide Tris-HCl mini-gels (Bio-Rad) and separated by SDS-PAGE. The volume of sample loaded was corrected for total cell protein per flask. Proteins were transferred electrophoretically to nitrocellulose membranes. The membranes were saturated with Blotto blocker (Pierce) and incubated overnight at 4° C. with 1:500 6E10 monoclonal antibody from Senetek. After washing, the membranes were incubated for 1 h at room temperature with the secondary antibody, alkaline phosphatase anti-mouse. IfG (Vector Laboratories) and developed using a BCIP/NBT subtrate kit (Vector Laboratories). The bands were quantitated by densitometric analyses (NIH Image, version 1.6) after obtaining a digital image of each membrane.

In FIG. 5, there is described benzolactam-induced secretion of sAPP in human fibroblasts. sAPP was quantified by densitometry after immunoblotting. Values from the BL treated cells were compared to those of untreated cells (set to 100%) within the same experiments. Bars represent the mean±s.d. of the data across cell lines. The value assigned to each cell line is the average of at least two replicates. In most cases, three to six separate experiments were performed per cell line. BL induced a significant dose dependent effect on sAPP secretion in AD fibroblasts (solid bars). The BL effect was completely abolished by staurosporine pre-treatment. Asterisks indicate statistical significance compared to basal values. Fibroblasts from age-matched controls (AC, open bars) also showed increased sAPP secretion after BL treatment, but only reached statistical significance at 10μM. Saturosporine also blacked the BL effect in these cell lines.

Digital images of all gels were obtained for quantitative analyses of the protein bands. The values from densitometric analyses were normalized as percentage increase over the basal secretion for each group of cells. BL treatment induced elevations in sAPP among AD cell lines at all three concentrations used in a dose-dependent manner, reaching an almost three-fold increase at 10μM as shown in FIG. 5 by the solid bars. The statistical analysis (one-way ANOVA) indicated an overall significance of p<0.0001 (F=19.2). The post test comparisons (Newman-Keuls) of basal levels of secretion for 1 and 10 μM BL treatment indicated a significance level of p<0.001, respectively. The elevation in sAPP secretion observed after 0.5 μM treatment did not reach statistical significance in the post test analysis. Pretreatment with staurosporine (ST) completely prevented the BL-induced sAPP secretion, further supporting the involvement of PKC. The level of secretion in ST-pretreated (FIG. 5) cells is significantly lower than corresponding concentrations of BL alone (p<0.01 and p<0.001 at 1 and 10 μM BL, respectively) and no different from basal levels or ST alone (p>0.05) in all comparisons, Newman-Keuls). Secretion of sAPP after treatment with 0.5 μM BL plus ST was virtually identical to basal levels and statistically no different than 0.5 μM BL alone (p>0.05 in both comparisons, Newman-Keuls).

The same experimental procedures and statistical analyses were applied to cell lines from age-matched controls. BL treatment also increased sAPP secretion in these cell lines (FIG. 5, open bars). This increase was less prominent than that observed for AD cells (one-way ANOVA p=0.001, F=4.043). Compared with basal levels, statistically significant differences were observed only at 10 μM BL (p<0.005, Newman-Keuls). A two-way ANOVA analyses confirmed significant differences due to treatment (p<0.0001). However, there was no effect of condition, i.e., no significant differences (p=0.39) between Alzheimer and control for sAPP secretion. Staurosporine was effective in preventing the increase in sAPP secretion caused by 10 μM BL in this group (p<0.05, 10 μM BL vs. 10 μM BL plus ST). Staurosporine alone was also without effect in controls (FIG. 5).

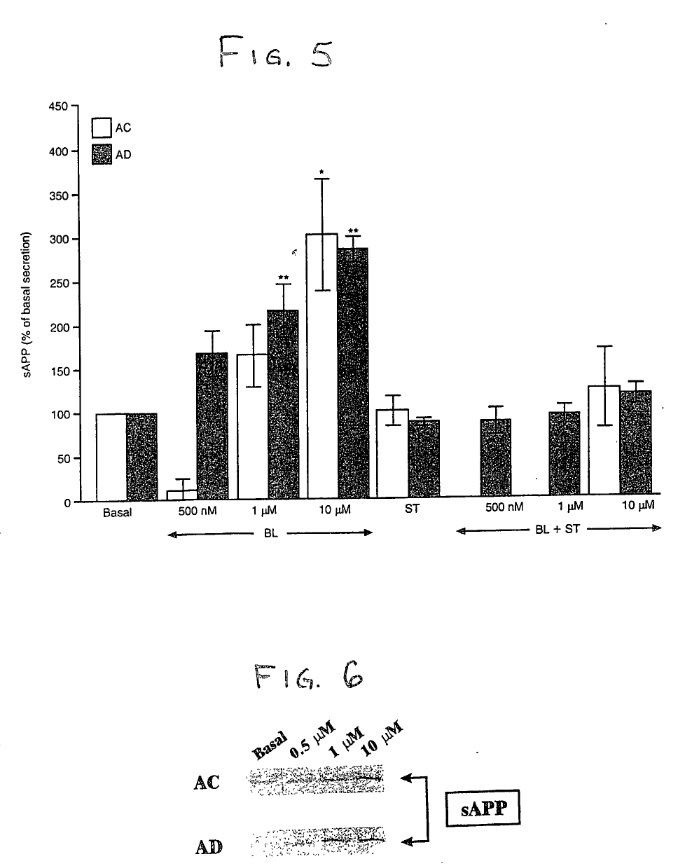

The sAPP basal levels of AD cell lines were slightly lower than in age-matched control cells, without reaching statistical significance (p=0.14), two tailed t-test). Representative immunoblots of control and AD cell lines are depicted in FIG. 6.

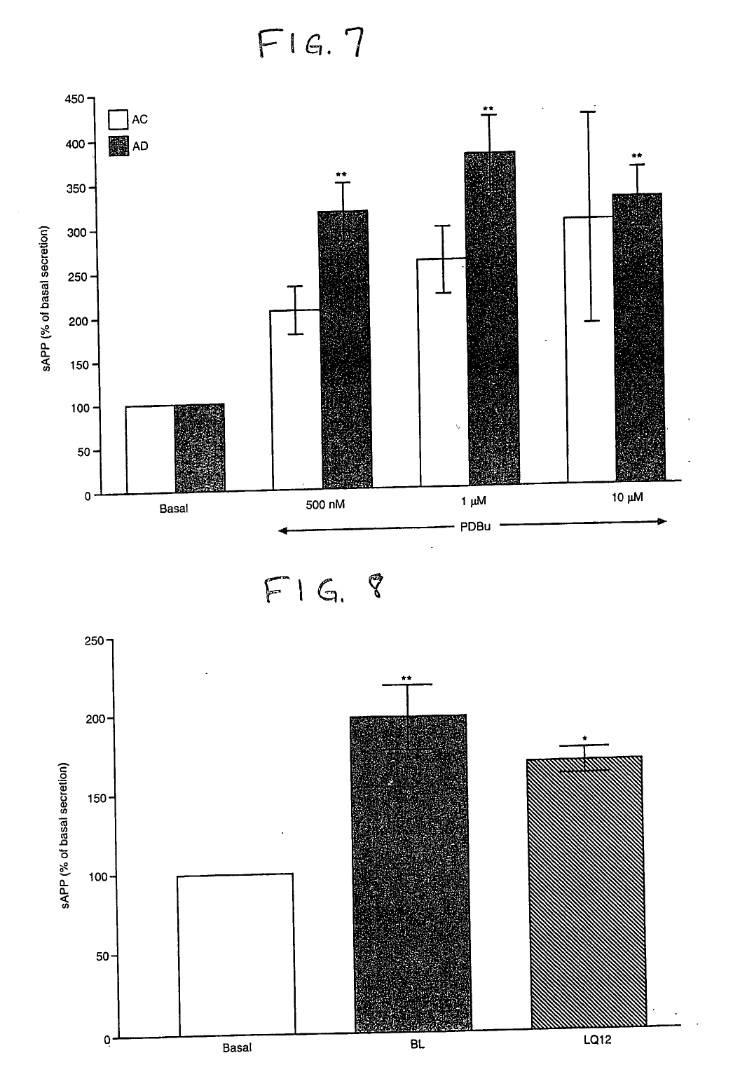

Phorbol ester treatment (same concentrations as BL) also induced significant increases in sAPP secretion in AD cell lines (one-way ANOVA, p=0.001, F=14.53). In fact, the effect is significantly higher than BL at 0.5 μM (BL vs. basal, p>0.05; PDBu vs. basal, p<0.01, Neuman-Keuls). Treatment with higher PDBu concentrations did not result in further significant elevations of sAPP secretion (FIG. 7). FIG. 7 in particular shows PDBu-induced secretion of sAPP in human fibroblasts. PDBu induced significant elevations of sAPP in AD (solid bars). The effect, unlike BL, was maximal at 500 nM and no further significant increases were observed at higher concentrations. All three different concentrations resulted in statistically significant increases (p<0.01, Newman-Keuls).

Although there was an elevation in sAPP secretion induced by PDBu in age-matched control cell lines, statistical significance was not reached (ANOVA, p=0.188, F=1.87).

Staurosporine pretreatment also significantly reduced the phorbol effect, although slightly less effectively than in the case of BL (data not shown).

Benzolactam and the related compound LQ12 (10 μM in both cases), also significantly increased sAPP secretion in PC12 cells (p=0.0002, one-way ANOVA). Compared with basal level, post-test analyses (Newman-Keuls) showed a significance level of p<0.001 and p<0.01 for BL and LQ12, respectively. These results are depicted in FIG. 8. In particular, FIG. 8 shows the effects of BL and LQ-12 on sAPP secretion in PC-12 cells. Densitometric values of sAPP on treated cells were compared with untreated controls for each experiment and repeated four times. Bars represent mean±s.d. Both compounds induced significant elevations of sAPP secretion. Asterisks indicated statistical significance compared to basal values (**p<0.001, *p<0.01).

These results confirm previous observations in cellular and animal models showing an important role for PKC involvement in APP metabolism, particularly in favoring the a-processing resulting in non-amyloidogenic sAPP. Increased secretion of sAPP is accompanied by a proportional reduction of the amyloidogenic fragments derived from the action of β and γ secretases. There are, however, two reports that show an increase in sAPP without changes in Aβ. These apparently contradictory results could be due to tissue-specific differences. In addition, Savage et al. reported phorbol-induced decrease in Aβ species without a noticeable increase in sAPP secretion in mouse brain.

Although we did not measure the proportional reduction of the amyloidogenic fragments here, there is ample experimental evidence from a number of independent laboratories supporting this assumption. Perhaps more importantly, we have provided evidence that these novel compounds, BL and LQ12, can be virtually as effective as the phorbol esters. These novel compounds have several advantages over phorbols. First, BL does not seem to be a tumor promoter; in fact recent evidence indicates that it shows antitumoral activity in rats (A. Kozikowski and R. Glazer, personal communication). They do not seem to saturate secretion of sAPP as PDBu does. BL produced an increase in sAPP at lower concentrations in AD than in the age-matched controls. Since it has been reported that AD-fibroblasts have a defective phorbol ester-stimulated secretion of sAPP, this marked effect of BL on AD fibroblasts constitutes another relevant advantage over phorbols. LQ12 is also a synthetic, more readily manipulable, chemical structure. B1 also has improved selectivity for isozymes that appears to be particularly defective in AD brain and peripheral tissues. In this regard, in a recent communication, Bhagavan et al. have shown that BL-induced significant α isozyme activation in AD fibroblasts. Moreover, this activation was linked to restoration of K⁺ channel activity and TEA-induced calcium elevations. Interestingly, BL but not phorbol was able to restore the normal molecular phenotype in fibroblasts. Also relevant to mention is an earlier finding showing that Aβ-₁₋₄₀, an APP subproduct of the non-a secretase alternative processing, inhibited normal K⁺ channel activity and TEA-induced calcium responses, mimicking the AD profile in fibroblasts. More recently it has been shown that Aβ₁₋₄₀ induces degradation of specific PKC isozymes, including α. Thus, all of these results taken together strongly suggest that PKC (perhaps with predominant involvement of specific isozymes) plays a significant role in AD pathophysiology, linking PP processing and molecular alterations. Isozyme specificity is also relevant in the sens that these compounds could potentially be modified towards an even greater y selectivity to eventually make them predominantly active in the brain, the organ primarily affected in AD. Consequently, this study provides initial evidence that targeting PKC isozymes may prove a useful approach towards developing compounds with significant impact in inhibiting excessive alternative amyloidogenic APP processing, thereby preventing or delaying subsequent cellular and eventually clinical manifestations of AD.

Novel PKC activators cause increased secretion of non-amyloidogenic sAPP in AD fibroblasts and PC12 cells. This elevated sAPP secretion may be also accompanied by a reduction of amyloidogenic fragments. These results further indicate a key role for PKC in APP processing and, therefore, in AD pathophysiology. The study also suggests that PKC may be a useful target for preventing or slowing the pathophysiological process in AD. Furthermore, these novel compounds offer the bases for drug design strategies targeted at PKC and APP processing that may significantly and beneficially alter the progression of this disease.

1. A method for increasing the generation of non-amyloidogenic soluble APP comprising activation of protein kinase C (PKC) by administering an effective amount of at least one PKC activator.

2. The method according to claim 1, wherein said PKC activator comprises a benzolactam.

3. The method according to claim 2, wherein said benzolactam comprises (2S, 5S)-8-(1′-decynyl)benzolactam or (2S, 5S)-8-(1′-decynyl)-7-methoxylbenzolactam.

4. A method for altering conditions associated with amyloid processing in order to enhance an α-secretase pathway to generate soluble α-amyloid precursor protein (α-APP) so as to prevent β-amyloid aggregation comprising administering an effective amount of a benzolactam.

5. The method according to claim 4, wherein the effective amount of said benzolactam is administered in vitro or in vivo.

6. The method according to claim 4, wherein the effective amount of said benzolactam is administered to a subject.

7. The method according to claim 6, wherein said subject is a rodent.

8. The method according to claim 4, wherein the effective amount of said benzolactam is administered to a biological sample.

9. The method according to claim 8, wherein said biological sample comprises a cell.

10. The method according to claim 4, wherein said benzolactam comprises (2S, 5S)-8-(1′-decynyl)benzolactam or (2S, 5S)-8-(1′-decynyl)-7-methoxylbenzolactam.

11. A method for reducing plaque formation caused by β-amyloid accumulation comprising administering an effective amount of a benzolactam.

12. The method according to claim 11, wherein said benzolactam comprises (2S, 5S)-8-(1′-decynyl)-benzolactam or (2S, 5S)-8-(1′-decynyl)-7-methoxylbenzolactam.

13. The method according to claim 11, wherein said administering is in vitro or in vivo.

14. The method according to claim 11, wherein the effective amount of said benzolactam is administered to a subject.

15. The method according to claim 14, wherein said subject is a rodent.

16. The method according to claim 11, wherein the effective amount of said benzolactam is administered to a biological sample.

17. The method according to claim 16, wherein said biological sample comprises a cell.

18. A method for treating Alzheimer's disease comprising activation of protein kinase C (PKC) by administering an effective amount of a benzolactam.
